(19) United States
(12) Patent Application Publication
(10) Pub. No.: US 2011/0230781 A1
(43) Pub. Date: Sep. 22, 2011

(54) APPARATUS AND METHOD FOR DEMENTIA DIAGNOSIS THROUGH EEG (ELECTROENCEPHALOGRAM) ANALYSIS

(76) Inventors: Seung Hwan Lee, Gyeonggi-do (KR); Chang Hwan Im, Gangwon-do (KR)

(21) Appl. No.: 13/119,600

(22) PCT Filed: Sep. 1, 2009
(86) PCT No.: PCT/KR09/04919

§ 371 Date: May 20, 2011

(30) Foreign Application Priority Data
Sep. 17, 2008 (KR) 10-2008-0091011

(51) Int. Cl. A61B 5/048

(52) U.S. Cl. 600/544

(57) ABSTRACT

The present invention provides an apparatus and a method for early diagnosis of dementia which measure and evaluate dimensional complexity or the generation of synchronized brain wave signals on a specific frequency or in a specific frequency band, based on the brain wave signals measured from a multi-channel brain wave measurement system. The apparatus comprises: a measurement unit with electrodes having plural channels for measuring brain wave signals; an amplification unit which amplifies brain wave signals measured by the measurement unit; a dementia diagnosis unit which measures dimensional complexity or the generation degree of synchronized brain wave signals on a specific frequency or in a specific frequency band based on the amplified brain wave signals; and a dementia determination unit which diagnoses dementia based on the dimensional complexity or the degree of synchronization measured in the dementia diagnosis unit.

### TECHNICAL FIELD

The present invention relates to an apparatus and method for dementia diagnosis by analyzing electroencephalogram or magnetoencephalography (EEG or MEG) (hereinafter, referred to as “brain wave”), and more particularly, to an apparatus and method for measuring dimensional complexity or the generation degree of synchronized brain wave signals on a specific frequency or in a specific frequency band based on the brain wave signals measured by a multi-channel brain wave measurement system and then diagnosing dementia based on the measured value.

### BACKGROUND ART

Aging of population is in rapid progress over the world, and accordingly, patients with dementia are rapidly increased. Thus, it is obvious that economical costs for managing and treating patients with dementia would increase in geometric progression.

In recent, remedies for delaying or improving dementia symptoms have been developed, and investments and endeavors for developing remedies with further improved effects continue without a break. However, the remedies are effective when being used at an early dementia stage, and thus early diagnosis and early treatment of dementia become a main issue.

In order to diagnose dementia, a dementia diagnosis method using psychological medical examination by interview is generally used. However, the dementia diagnosis method using psychological medical examination by interview takes several days and a lot of costs for checking and diagnosing a dementia state. Further, the above dementia diagnosis method using psychological medical examination by interview has a disadvantage in that an earlier state of dementia cannot be diagnosed before a clinical symptom appears.

Other conventional techniques currently studied for dementia diagnosis include a method for diagnosing the degree of dementia by photographing a brain image using an fMRI or PET, a bio-marker method for diagnosing the degree of dementia by analyzing a blood or cerebrospinal fluid extracted from a patient with dementia, and a method for dementia diagnosis by measuring brain waves (EEG or MEG).

In an earlier dementia state, the abnormal phenomena of acetylcholine-related functions in the brain were proved with many experimental evidences, and the close relationship between acetylcholine and beta amyloid was also experimentally checked. Techniques capable of diagnosing dementia at an earlier stage by measuring a change of beta amyloid or acetylcholine in the brain by a brain image photographing technique using PET are under development. However, even after the technique concerning the brain image photographing method was completely developed, the technique did not spread widely due to expensive equipment.

There are disadvantages in that the bio-marker method is accompanied with the pain of a dementia patient since a blood or cerebrospinal fluid is directly extracted from him or her, and a lot of time is consumed for diagnosing the degree of dementia by analyzing the extracted blood or cerebrospinal fluid.

Meanwhile, in the method for diagnosing dementia by measuring brain waves (EEG or MEG), background brain waves or relaxation brain waves of a dementia patient are measured, and the measured brain waves are compared with background or relaxation brain waves of a normal person and then dementia of the patient is diagnosed. The background or relaxation brain waves mean brain waves when a person sits still on a chair with the eyes closed. In the conventional art, the measured brain waves are classified into delta (0 to 4 Hz), theta (4 to 8 Hz), alpha (8 to 12 Hz), beta (15 to 30 Hz), and gamma (30 to 60 Hz) bands depending on frequency components.

However, the principle of generation and physiological meaning and roles of frequency components in the delta, theta, alpha, beta, and gamma bands are not well known in the art until now. Thus, in the method for dementia diagnosis of a dementia patient by measuring brain waves, frequency components in every band are considered as independent component regardless of the behavior of a dementia patient, and in a general case, frequency components in the theta band among the background brain waves or the relaxation brain waves of a dementia patient are compared with frequency components in the theta band of background brain waves or relaxation brain waves of a normal person to diagnose dementia of the dementia patient.

FIG. 1 shows brain waves of a dementia patient having the degree of dementia in a severe or more serious level and brain waves of a normal person, both measured in a relaxed state.

In FIG. 1 (a), a lower waveform shows brain waves of a patient having the degree of dementia in a severe or more serious level, measured on a time axis while he or she is relaxing with the eyes closed, and an upper waveform shows brain waves of a normal person. Also, FIG. 1 (b) shows frequency components 1 in the theta band of a patient having the degree of dementia in a severe or more serious level and frequency components 2 in the theta band of a normal person, which are measured on a frequency axis in a relaxed state with the eyes closed.

As shown in FIG. 1 (a) and FIG. 1 (b), it could be understood that the magnitude of the frequency components in the theta band of a patent having the degree of dementia in a severe or more serious level is greater than the magnitude of the frequency components in the theta band of a normal person, which are measured in a relaxed state with the eyes closed.

Meanwhile, FIG. 2 shows brain waves of an early dementia patient, measured in a relaxed state with the eyes closed, and brain waves of a normal person, measured in a relaxed state with the eyes closed.

In FIG. 2 (a), a lower waveform shows brain waves of an early dementia patient, measured on a time axis while he or she is relaxing with the eyes closed, and an upper waveform shows brain waves of a normal person. Also, FIG. 2 (b) shows frequency components 1 in the theta band of an early dementia patient and frequency components 2 in the theta band of a normal person, which are measured on a frequency axis in a relaxed state with the eyes closed.

As shown in FIG. 2 (a) and FIG. 2 (b), it could be understood that the magnitude of the frequency components in the theta band of an early dementia patent is not greatly different from the magnitude of the frequency components in the theta band of a normal person, which are measured in a relaxed state with the eyes closed.

As mentioned above, since the frequency components in the theta band of an early dementia patient are not greatly different from the frequency components in the theta band of a normal person, the method of diagnosing dementia by measuring brain waves (EEG or MEG) cannot diagnose dementia at an early stage. Also, since dementia remedies recently developed give effects when being used at an early stage of dementia, the above problem may be considered as a very serious issue in the treatment of dementia. Thus, there is urgently needed to develop a method for dementia diagnosis at an early stage.

### DISCLOSURE

### Technical Problem

According to an aspect of the present invention for achieving the objects, there is provided an apparatus for dementia diagnosis through brain wave analysis, which includes a measurement unit with electrodes having a plurality of channels for measuring brain wave signals; an amplification unit for amplifying the brain wave signals measured by the measurement unit; a dementia diagnosis unit for measuring dimensional complexity or the generation degree of synchronized brain wave signals on a specific frequency or in a specific frequency band based on the amplified brain wave signals; and a dementia determination unit for diagnosing dementia based on the dimensional complexity or the generation degree of synchronization measured in the dementia diagnosis unit.

Preferably, the apparatus further includes a memory unit for providing specific frequency information or specific frequency band information for measuring the synchronization in the dementia diagnosis unit, and providing at least one of MMSE (Mini Mental Status Examination), GDS (Global Deterioration Scale), DOI (Duration of Illness), and CDR (Clinical Dementia Rating) information to the dementia determination unit.

Preferably, the dementia diagnosis unit measures the synchronization of the brain wave signals using any one of GFS (Global Field Synchronization) and GSI (Global Synchronization Index).

According to another aspect of the present invention for achieving the objects, there is provided a method for dementia diagnosis through brain wave analysis, which comprises the steps of measuring brain wave signals by performing potential measurement based on neuronal activation in the brain at fixed sampling intervals; measuring the generation of synchronized brain wave signals on a specific frequency based on the measured brain wave signals; and determining that a diagnosed person as a dementia patient if it is determined as a result of the measurement that no synchronized brain wave signal is generated.

Preferably, GFS and GSI are used for measuring the synchronization of the brain signals.

Preferably, the method further comprises the steps of classifying the diagnosed person determined as a dementia patient into a dementia-suspected group; re-determining brain wave signals of the classified dementia-suspected group by using any one of MMSE, GDS, and CDR information, which determine the degree of dementia on a specific frequency or in a specific frequency band according to an existing method; and as a result of the re-determination, determining the diagnosed person as a dementia-suspected patient if it is determined that the diagnosed person shows a result corresponding to a normal person and finally determining the diagnosed person as a dementia patient if it is determined that the diagnosed person shows a result corresponding to a dementia patient.

Preferably, the method of measuring the synchronization of the brain signals comprises the steps of performing Fourier transform to the brain wave signals measured at an electrode of each channel to calculate a complex number value at a specific frequency (f); making the calculated complex number value correspond to a complex plane to perform PCA (Principal Component Analysis) for distribution of points on the complex plane so that two eigenvalues (E₁ and E₂) are extracted; applying the calculated eigenvalues (E₁ and E₂) to an equation

$$GFS ( f ) = \frac{\vert {E ( f )}_{1} - {E ( f )}_{2} \vert}{{E ( f )}_{1} + {E ( f )}_{2}}$$

to calculate a GFS value; and determining that the measured brain wave signals are synchronized if the calculated GFS value is closer to 1, rather than to 0, and determining that the measured brain wave signals are not synchronized if the calculated GFS value is closer to 0, rather than to 1.

Preferably, the method for synchronization of the brain wave signals comprises the steps of calculating synchronization of electrode pairs corresponding to all brain wave signals measured on a specific frequency and combining the synchronization into a matrix format; analyzing a pattern of the synchronization matrix through eigenvalue decomposition of the combined matrix to extract eigenvalues; applying a greatest value (80 ) of the extracted eigenvalues to an equation |GSI=(λ−ν)/(M−ν)| to calculate a GSI value; and determining that the measured brain wave signals are synchronized if the calculated GFI value is closer to 1, rather than to 0, and determining that the measured brain wave signals are not synchronized if the calculated GFI value is closer to 0, rather than to 1, wherein M of the equation represents the number of the measured brain wave signals (which is equal to the number of electrodes), and ν is an average eigenvalue according to eigenvalue calculation using surrogate data.

According to further aspect of the present invention for achieving the objects, there is provided a method for dementia diagnosis through brain wave analysis, which comprises the steps of measuring brain wave signals by performing potential measurement based on neuronal activation in the brain at fixed sampling intervals; measuring dimensional complexity in the gamma band based on the measured brain wave signals; and as a result of the measurement, determining a diagnosed person as a dementia patient if the measured dimensional complexity is greater than a threshold dimensional complexity at which dimensional complexity is defined to a minimal value.

Preferably, the method further comprises the steps of classifying the diagnosed person determined as a dementia patient into a dementia-suspected group; re-determining brain wave signals of the classified dementia-suspected group by using any one of MMSE, GDS, and CDR information, which determine the degree of dementia in a gamma band according to an existing method; and as a result of the re-determination, determining the diagnosed person as a dementia-suspected patient if it is determined that the diagnosed person shows a result corresponding to a normal person and finally determining the diagnosed person as a dementia patient if it is determined that the diagnosed person shows a result corresponding to a dementia patient.

Preferably, the dimensional complexity (D2) is measured using an equation

$$D 2 = {lim}_{r -> 0} {lim}_{N -> ∞} \frac{log C ( r , N )}{log r} ,$$

where C(r,N) is defined using an equation

$$C ( r , N ) = \frac{2}{( N - W ) ( N - 1 - W )} {\sum }_{i = 1}^{N} {\sum }_{j = i + 1 + W}^{N} θ ( r - \vert {{x}^{->}}_{i} - {{x}^{->}}_{j} \vert ) ,$$

xᵢ and xⱼ are points of a path in a phase space, N is a data pointer number in the phase space, the distance r is a range surrounding each reference point xᵢ, and θ is a Heaviside function defined as 0 if x<0 and 1 if x≧0.

### ADVANTAGEOUS EFFECTS

The apparatus and method for dementia diagnosis through brain wave analysis according to the present invention as described above give the following effects.

First, it is possible to provide information allowing early dementia diagnosis or to allow early dementia diagnosis by diagnosing dementia based on dimensional complexity or the generation degree of synchronized brain wave signals on a specific frequency or in a specific frequency band based on measured brain wave signals.

Second, the degree of dementia may be more accurately measured and evaluated since the degree of dementia is determined through re-determination using MMSE (Mini Mental Status Examination), GDS (Global Deterioration Scale), DOI (Duration of Illness), or CDR (Clinical Dementia Rating) information among the dementia-suspected group classified by GFS or GSI.

Third, since dementia is diagnosed by measuring brain waves of a dementia patient, the stage of dementia may be diagnosed without giving any pain to the dementia patient.

### DESCRIPTION OF DRAWINGS

FIG. 1 is a waveform and a graph showing brain waves of a dementia patient having the degree of dementia in a severe or more serious level and brain waves of a normal person, measured in a relaxed state, both measured in a relaxed state.

FIG. 2 is a waveform and a graph showing brain waves of an early dementia patient, measured in a relaxed state with the eyes closed, and brain waves of a normal person, measured in a relaxed state with the eyes closed.

FIG. 3 is a block diagram showing an apparatus for dementia diagnosis through brain wave analysis according to an embodiment of the present invention.

FIG. 4 is a flowchart for illustrating a method for dementia diagnosis through brain wave analysis according to an embodiment of the present invention.

FIG. 5 is a flowchart for illustrating a method for measuring synchronization of brain wave signals using GFS.

FIG. 6 is a flowchart for illustrating a method for measuring synchronization of brain wave signals using GSI.

FIG. 7 is a graph showing distribution of points in a complex plane, which correspond to complex number values expressed on a specific frequency for illustrating the synchronization through a GFS value.

FIG. 8 is a flowchart for illustrating a method for dementia diagnosis through brain wave analysis according to another embodiment of the present invention.

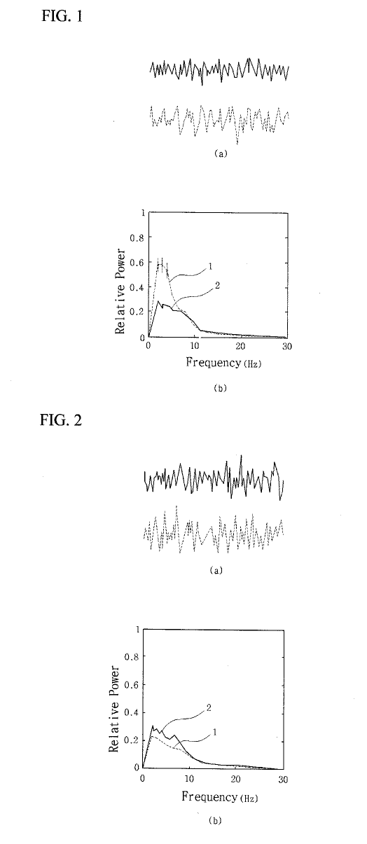

### BEST MODE

Other objects, features, and advantages of the present invention will be obvious through the detailed description of embodiments with reference to the accompanying drawings.

A preferred embodiment of a method for dementia diagnosis using brain wave analysis according to the present invention will be described as follows with reference to the accompanying drawings. However, the present invention is not limited to the following embodiments but may be implemented in various ways, and the following embodiments are just given for perfect disclosure of the present invention and better understanding of the scope of the present invention to those having ordinary skill in the art.

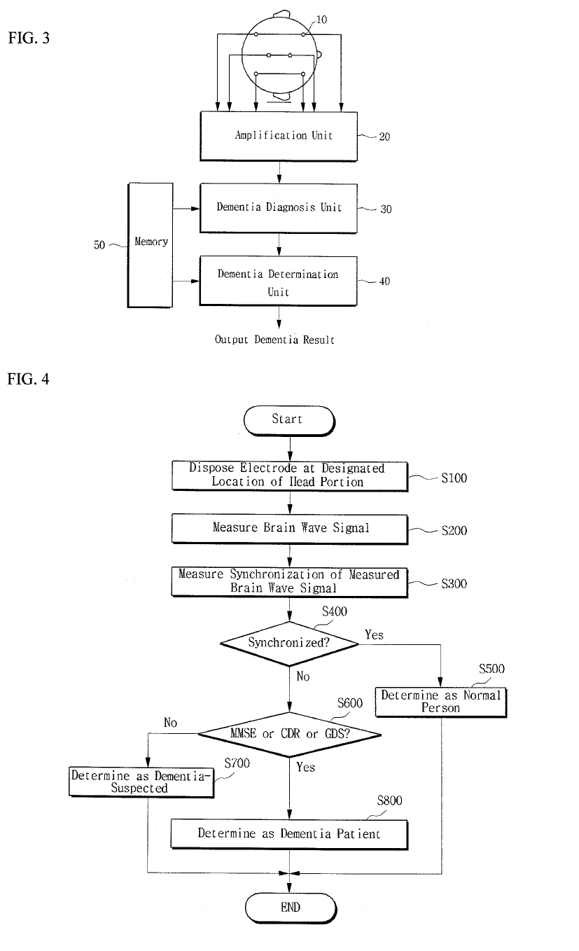

FIG. 3 is a block diagram showing an apparatus for dementia diagnosis through brain wave analysis according to an embodiment of the present invention.

As shown in FIG. 3, the dementia diagnosis apparatus includes a measurement unit 10 with electrodes having a plurality of channels for measuring brain wave signals, an amplification unit 20 for amplifying the brain wave signals measured by the measurement unit 10, a dementia diagnosis unit 30 for measuring dimensional complexity or the generation degree of synchronized brain wave signals on a specific frequency or in a specific frequency band based on the amplified brain wave signals, and a dementia determination unit 40 for diagnosing dementia based on the dimensional complexity or the generation degree of synchronization measured by the dementia diagnosis unit 30. Also, the dementia diagnosis apparatus further includes a memory unit 50 providing specific frequency information or specific frequency band information for measuring the dimensional complexity or the degree of synchronization by the dementia diagnosis unit 30 and also providing MMSE, GDS, DOI, or CDR information to the dementia determination unit 40.

At this time, the dementia diagnosis unit 30 measures the synchronization of brain wave signals using any one of GFS and GSI, and measures the dimensional complexity using the brain wave signals in a gamma band (30 to 70 Hz) among the brain wave signals measured in the measurement unit 10.

The operation of the dementia diagnosis apparatus using brain wave analysis according to the present invention as configured above will be described below in detail with reference to the accompanying drawings.

### FIRST EMBODIMENT

FIG. 4 is a flowchart for illustrating a method for dementia diagnosis through brain wave analysis according to an embodiment of the present invention.

Referring to FIG. 4, firstly, the measurement unit 10 having electrodes is disposed at a designated location of a head portion (S100), and then, brain wave signals are measured by performing potential measurement based on neuronal activation in the brain at fixed sampling intervals (S200). The electrodes are attached to a head skin of a dementia patient in accordance with the International Standard 10-20, or a brain wave measurement cap having the electrodes attached thereto is worn on the head of the dementia patient to measure brain waves (EEG or MEG) of the dementia patient. At this time, the brain waves measured by the measurement unit are weak signals, and thus, the measured brain wave signals are amplified by the amplification unit 20.

Subsequently, based on the brain wave signals measured at the electrodes of each channel, the generation degree of synchronized brain wave signals on a specific frequency or in a specific frequency band which is provided from the memory unit 50 is measured through the dementia diagnosis unit 30 (S300). At this time, as a method for measuring the synchronization of the brain wave signals, GFS and GSI are used.

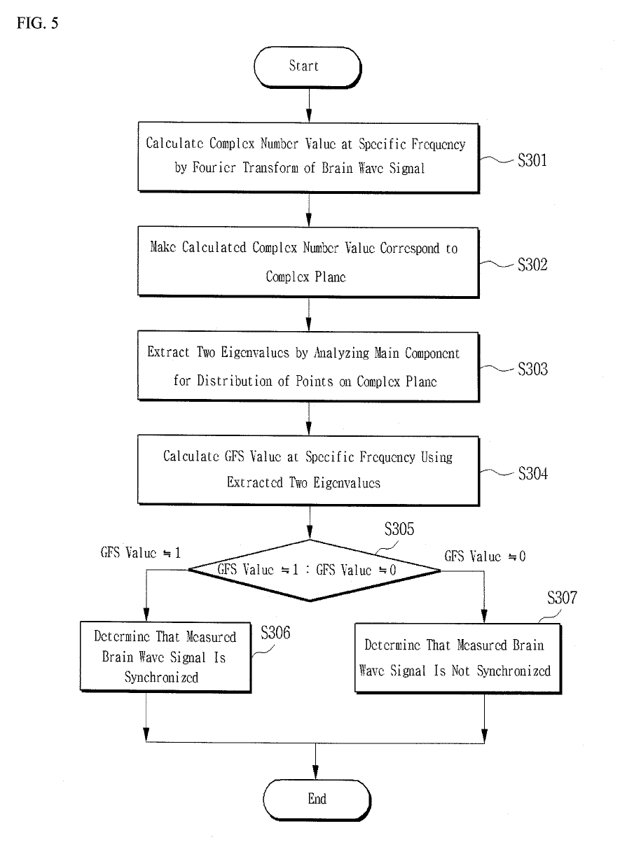

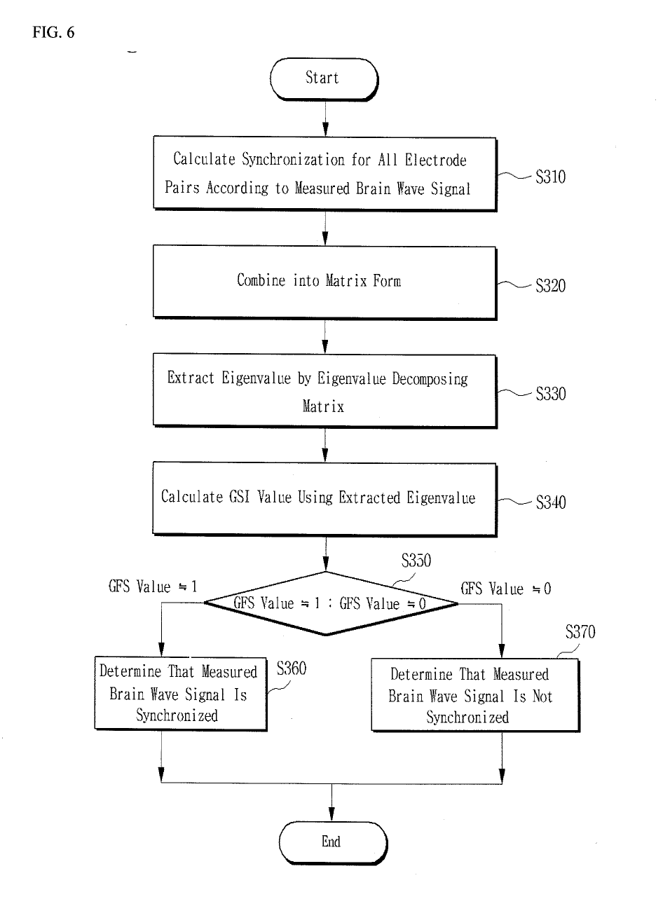

FIG. 5 is a flowchart for illustrating a method for measuring the synchronization of brain wave signals by using GFS, and FIG. 6 is a flowchart for illustrating a method for measuring the synchronization of brain wave signals by using GSI.

GFS is an index initially proposed by Koenig et al. in 2001 (Koenig, T., Lehmann, D., Saito, N., Kuginuki, T., Kinoshita, T., Koukkou, M., 2001. Decreased functional connectivity of EEG theta-frequency activity in first-episode, neuroleptic-naïve patients with schizophrenia: preliminary results. Schizophr. Res. 50, 55-60.), and as shown in FIG. 5, in GFS, the brain wave signals measured at the electrodes of each channel are converted into a frequency domain by means of Fourier transform and expressed as a complex number value on a specific frequency (S301).

The specific frequency used at this time is information for determining the degree of dementia at each frequency, which may be shown as in the following Table 1. This is stored in the memory unit 50 in advance.

TABLE 1

| Band [symbol] | AD patients (N = 22) [symbol] | Controls (N = 23) [symbol] | P [symbol] |
|---|---|---|---|
| Delta(1-3 Hz) [symbol] | 0.547 ± 0.040 [symbol] | 0.561 ± 0.036 [symbol] | 0.190 [symbol] |
| Theta(4-7 Hz) [symbol] | 0.540 ± 0.023 [symbol] | 0.555 ± 0.032 [symbol] | 0.190 [symbol] |
| Alpha(8-12 Hz) [symbol] | 0.560 ± 0.055 [symbol] | 0.575 ± 0.037 [symbol] | 0.295 [symbol] |
| Beta1(13-18 Hz) [symbol] | 0.499 ± 0.026 [symbol] | 0.523 ± 0.020 [symbol] | 0.001 [symbol] |
| Beta2(19-21 Hz) [symbol] | 0.496 ± 0.027 [symbol] | 0.514 ± 0.029 [symbol] | 0.035 [symbol] |
| Beta3(22-30 Hz) [symbol] | 0.482 ± 0.021 [symbol] | 0.505 ± 0.026 [symbol] | 0.002 [symbol] |
| Gamma(30-50 Hz) [symbol] | 0.475 ± 0.041 [symbol] | 0.486 ± 0.027 [symbol] | 0.280 [symbol] |
| Full(1-70 Hz) [symbol] | 0.498 ± 0.019 [symbol] | 0.514 ± 0.018 [symbol] | 0.005 [symbol] |

At this time, since the value of P (a meaningful probability value according to statistics) is reliable when being equal to or smaller than 0.05, frequency components in theta, beta1, beta2, beta3, and full bands are used as the specific frequency to thereby determine the degree of dementia.

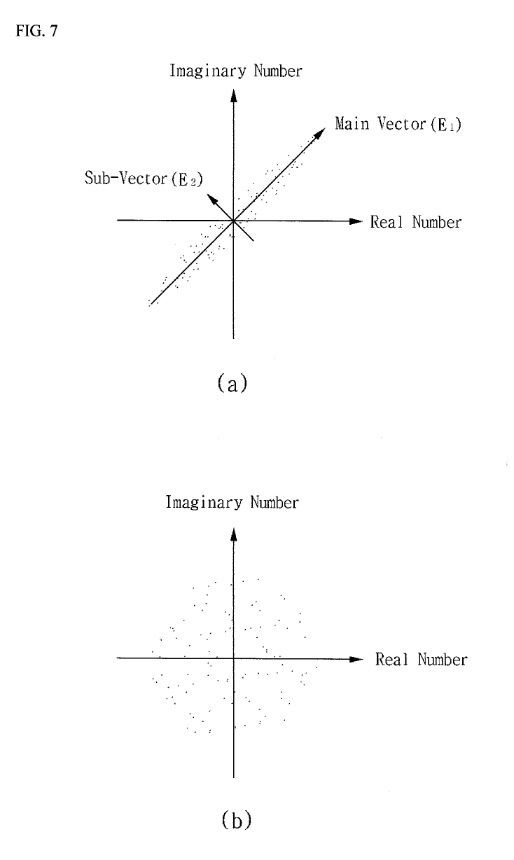

When the complex number value expressed on the specific frequency are set to correspond to a point in a complex plane, a direction component of each vector means the phase of the signal at the specific frequency. Also, if phase values at various electrodes have synchronized properties, the distribution of points expressed on the complex plane would have a certain directionality as shown in FIG. 7 (a), but, if not synchronized, the distribution of points on the complex plane would have no directionality as shown in FIG. 7 (b).

As a method for measuring the distribution of points, first, calculated complex number values are set to correspond to points in a complex plane (S302), and subsequently, principal component analysis (PCA) is performed on the distribution of points on the complex plane to extract two eigenvalues (S303).

For reference, assuming that eigenvalues at the specific frequency are E₁ and E₂, E₁ is an eigenvalue representing a most major direction component, and E₂ means an eigenvalue of a direction component perpendicular to the E₁. A GFS value at the specific frequency f is calculated using the following Equation 1.

$$GFS ( f ) = \frac{\vert {E ( f )}_{1} - {E ( f )}_{2} \vert}{{E ( f )}_{1} + {E ( f )}_{2}} \qquad Equation 1$$

As in Equation 1, if points are distributed on a straight line as shown in FIG. 7 (a), E₂ that is a direction component perpendicular to the major direction component would have a value substantially close to 0, and thus, the GFS value would have a value close to 1. However, when points are distributed without regularity and dispersed uniformly as shown in FIG. 7 (b), the values of E₁ and E₂ would have substantially the same, and thus the GFS value would be a value close to 0.

As mentioned above, the fact that the GFS value calculated by Equation 1 is a value close to 0 means that the brain waves measured at all electrodes are synchronized (S306), and the fact that the GFS value is a value close to 0 means that the measured brain waves are not synchronized since there is no common phase (S307).

In addition, GSI is an index initially proposed by Li et al. in 2007 (Li, X., Cui, D., Jiruska, P., Fox, J. E., Yao, X., Jefferys, J. G. R., 2007. Synchronization Measurement of Multiple Neuronal Populations. J. Neurophysiol, 98, 3341-3348.), and as shown in FIG. 6, in GSI, synchronization is calculated for electrode pairs corresponding to all brain wave signals measured at the specific frequency (S310), and then, the calculated synchronizations are combined in a matrix form (S320). At this time, the method for calculating the synchronization is performed by applying an existing method such as coherence, phase coherence, equal-time correlation, and so on.

The specific frequency used at this time is information for determining the degree of dementia at each frequency, which may be shown as in the following Table 2. This information is stored in the memory unit 50 in advance.

TABLE 2

| | AD (N = 22) | | Controls (N = 23) | | |
|---|---|---|---|---|---|
| Band | Mean | SD | Mean | SD | P |
| Delta(1-3 Hz) | 0.28 | 0.03 | 0.29 | 0.05 | 0.199 |
| Theta(4-7 Hz) | 0.28 | 0.03 | 0.31 | 0.04 | 0.017 |
| Alpha(8-12 Hz) | 0.34 | 0.09 | 0.34 | 0.05 | 0.899 |
| Beta1(13-18 Hz) | 0.24 | 0.03 | 0.28 | 0.03 | 0.000 |
| Beta2(19-21 Hz) | 0.22 | 0.03 | 0.26 | 0.03 | 0.000 |
| Beta3(22-30 Hz) | 0.22 | 0.02 | 0.26 | 0.03 | 0.000 |
| Gamma(30-50 Hz) | 0.21 | 0.03 | 0.25 | 0.03 | 0.000 |

At this time, since the value of P (a meaningful probability value according to statistics) is reliable when being equal to or smaller than 0.05, frequency components in theta, beta1, beta2, beta3, and gamma bands are used as the specific frequency to thereby determine the degree of dementia. An element of a matrix made by a combination in the matrix form through a specific frequency as mentioned above generally has a value between −1 and 1, wherein an element having a value close to 1 means that great synchronization occurs between corresponding two brain wave signals, and an element having a value close to −1 means that synchronization hardly occur between two brain wave signals. Generally, the pattern of this matrix has a locally-clustered appearance, which means that synchronization occurs between specific regions of the brain.

Thus, the pattern of the synchronization matrix is analyzed through eigenvalues decomposition of the matrix to thereby extract eigenvalues (S330).

At this time, it could be understood that synchronization occurs more greatly between difference regions of the brain as an eigenvalue having a greatest value among the extracted eigenvalues is greater. Using this property, the GSI value is calculated using the following Equation 2 (S340).

Equation 2

GSI=(λ−ν)/(M−ν)

In Equation 2, M means the number of measured brain wave signals (which is equal to the number of electrodes), and λ means a greatest eigenvalue in the generated synchronization matrix. Since it should be considered whether the eigenvalue of the synchronization matrix is a statistically meaningfully great value, the eigenvalue is calculated in the same way for surrogate data generated by randomly deforming the phase of the measured brain wave signals, and this process is repeated to obtain an average eigenvalue. This value is referred to as ν.

The GSI value calculated using Equation 2 has a value between 0 and 1. The GSI value closer to 1 means that the brain waves measured at all channels are synchronized (S360) while the GSI value closer to 0 means that the synchronization of the brain waves is deteriorated (S370). For reference, the brain wave signals measured at the electrodes of each channel are substantially locally synchronized, and thus the correlation characteristics may be extracted more easily by using the synchronization measured using GSI, rather than GFS.

As mentioned above, whether synchronization is performed well at a specific frequency is standardized through the GFS or GSI, and then, a diagnosed person is determined as a normal person if the synchronization is performed (S500).

In addition, in a case where the synchronization is not performed by being standardized through the GFS or GSI, the diagnosed person is classified into a dementia-suspected group. Also, for the diagnosed person classified into the dementia-suspected group, re-determination is performed using at least one of MMSE, GDS, and CDR information, which determine the degree of dementia on a specific frequency or in a specific frequency band, which is stored in the memory unit 50 and calculated in an existing method (S600). For reference, MMSE, GDS, or CDR information for determining the degree of dementia at each aforementioned frequency may be expressed as in the following Table 3. It is stored in the memory unit 50 in advance.

TABLE 3

| Band [symbol] | MMSE(r) [symbol] | P [symbol] | CDR(r) [symbol] | P [symbol] |
|---|---|---|---|---|
| Delta(1-3 Hz) [symbol] | 0.09 [symbol] | 0.562 [symbol] | −0.07 [symbol] | 0.641 [symbol] |
| Theta(4-7 Hz) [symbol] | 0.07 [symbol] | 0.652 [symbol] | −0.16 [symbol] | 0.287 [symbol] |
| Alpha(8-12 Hz) [symbol] | 0.35 [symbol] | 0.018 [symbol] | −0.32 [symbol] | 0.032 [symbol] |
| Beta1(13-18 Hz) [symbol] | 0.49 [symbol] | 0.001 [symbol] | −0.49 [symbol] | 0.001 [symbol] |
| Beta2(19-21 Hz) [symbol] | 0.46 [symbol] | 0.001 [symbol] | −0.39 [symbol] | 0.009 [symbol] |
| Beta3(22-30 Hz) [symbol] | 0.52 [symbol] | 0.0002 [symbol] | −0.47 [symbol] | 0.001 [symbol] |
| Gamma(30-50 Hz) [symbol] | 0.25 [symbol] | 0.092 [symbol] | −0.22 [symbol] | 0.155 [symbol] |
| Full(1-70 Hz) [symbol] | 0.46 [symbol] | 0.001 [symbol] | −0.42 [symbol] | 0.004 [symbol] |

At this time, since the value of P (a meaningful probability value according to statistics) is reliable when being equal to or smaller than 0.05, the degree of dementia is determined using frequency components in alpha, beta1, beta2, beta3, and full bands in a case where MMSE information is used, and the degree of dementia is determined using frequency components in alpha, beta1, beta2, beta3, and full bands in a case where CDR information is used.

In addition, the dementia-suspected group classified through the GFS or GSI is re-determined using MMSE, CDS, or CDR information (S600), and as a result, the diagnosed person is determined as a dementia-suspected patient if the diagnosed person has a value corresponding to a normal person (S700). Also, as a result of the re-determination (S600), the diagnosed person is finally determined as a dementia patient if the diagnosed person has a point corresponding to dementia (S800).

### SECOND EMBODIMENT

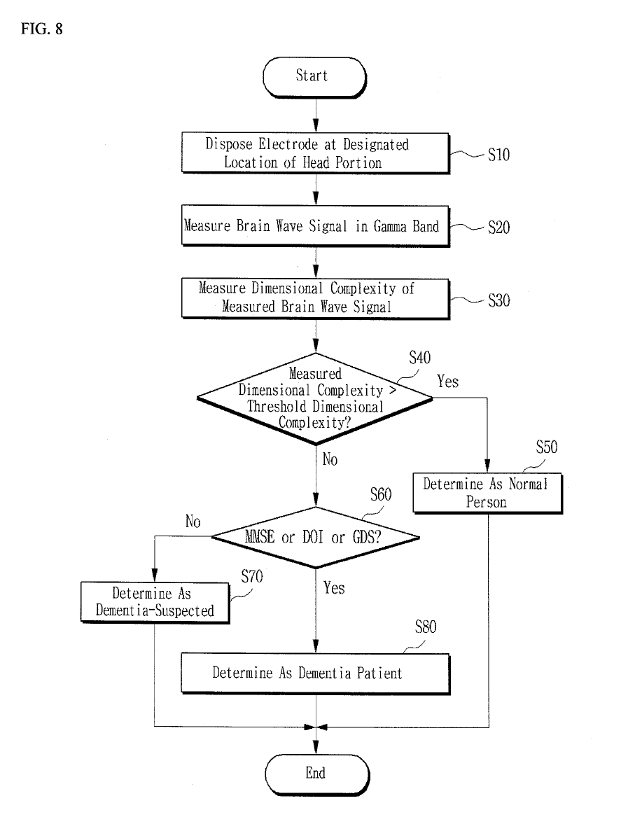

FIG. 8 is a flowchart for illustrating a method for dementia diagnosis through brain wave analysis according to another embodiment of the present invention.

Referring to FIG. 8, firstly, the measurement unit 10 having electrodes is disposed at a designated location of a head portion (S10), and then, brain wave signals in a gamma band (30 to 70 Hz) are measured by performing potential measurement based on neuronal activation in the brain at fixed sampling intervals (S20). The electrodes are attached to a head skin of a dementia patient in accordance with the International Standard 10-20, or a brain wave measurement cap having the electrodes attached thereto is worn on the head of the dementia patient to measure brain waves (EEG or MEG) of the dementia patient. At this time, the brain waves measured by the measurement unit are weak signals, and thus, the measured brain wave signals are amplified by the amplification unit 20.

Subsequently, based on the brain wave signals measured at the electrodes of each channel, dimensional complexity in the gamma band, which is provided from the memory unit 50 is measured through the dementia diagnosis unit 30 (S30).

At this time, a method for measuring the dimensional complexity D2 of the brain wave signal uses the following Equation 3.

$$D 2 = {lim}_{r -> 0} {lim}_{N -> ∞} \frac{log C ( r , N )}{log r} \qquad Equation 3$$

Also, C(r,N) is defined as in the following Equation 4.

$$C ( r , N ) = \frac{2}{( N - W ) ( N - 1 - W )} {\sum }_{i = 1}^{N} {\sum }_{j = i + 1 + W}^{N} θ ( r - \vert {{x}^{->}}_{i} - {{x}^{->}}_{j} \vert ) \qquad Equation 4$$

At this time, xᵢ and xⱼ are points of a path in a phase space, N is a data pointer number in the phase space, the distance r is a range surrounding each reference point xᵢ, and θ is a Heaviside function defined as 0 if x<0 and 1 if x≧0.

In addition, the information for determining the degree of dementia in the gamma band, which is used in the dementia diagnosis unit 30, may be expressed as in the following Table 4. It is stored in the memory unit 50 in advance.

TABLE 4

| | | AD [symbol] | | NC [symbol] | | Mann Whitney | |
|---|---|---|---|---|---|---|---|
| [symbol] | | (N = 24) [symbol] | | (N = 18) [symbol] | | Test [symbol] | |
| [symbol] | Cortical region [symbol] | Mean [symbol] | SD [symbol] | Mean [symbol] | SD [symbol] | Z [symbol] | P [symbol] |
| [symbol] | LF [symbol] | 4.33 [symbol] | 0.91 [symbol] | 4.45 [symbol] | 1.2 [symbol] | −0.89 [symbol] | 0.374 [symbol] |
| [symbol] | LT [symbol] | 4.03 [symbol] | 0.89 [symbol] | 4.57 [symbol] | 0.6 [symbol] | −2.05 [symbol] | 0.041 [symbol] |
| [symbol] | LP [symbol] | 4.65 [symbol] | 0.9 [symbol] | 5.07 [symbol] | 0.63 [symbol] | −1.07 [symbol] | 0.286 [symbol] |
| Gamma band [symbol] | LO [symbol] | 4.75 [symbol] | 0.74 [symbol] | 4.78 [symbol] | 0.84 [symbol] | −0.09 [symbol] | 0.929 [symbol] |
| | RF [symbol] | 4.51 [symbol] | 0.89 [symbol] | 4.77 [symbol] | 0.98 [symbol] | −1.07 [symbol] | 0.242 [symbol] |
| | RT [symbol] | 4.23 [symbol] | 0.94 [symbol] | 4.79 [symbol] | 0.67 [symbol] | −2.36 [symbol] | 0.018 [symbol] |
| | RP [symbol] | 4.97 [symbol] | 0.67 [symbol] | 5.02 [symbol] | 0.51 [symbol] | −0.43 [symbol] | 0.666 [symbol] |
| | RO [symbol] | 4.59 [symbol] | 0.94 [symbol] | 5.13 [symbol] | 0.56 [symbol] | −2.19 [symbol] | 0.029 [symbol] |
| [symbol] | LF [symbol] | 4.51 [symbol] | 0.46 [symbol] | 4.39 [symbol] | 0.57 [symbol] | −0.38 [symbol] | 0.703 [symbol] |
| [symbol] | LT [symbol] | 4.38 [symbol] | 0.56 [symbol] | 4.21 [symbol] | 0.77 [symbol] | −0.39 [symbol] | 0.694 [symbol] |
| [symbol] | LP [symbol] | 4.58 [symbol] | 0.43 [symbol] | 4.43 [symbol] | 0.66 [symbol] | −0.56 [symbol] | 0.576 [symbol] |
| Whole band [symbol] | LO [symbol] | 4.62 [symbol] | 0.53 [symbol] | 4.34 [symbol] | 0.89 [symbol] | −0.69 [symbol] | 0.493 [symbol] |
| | RF [symbol] | 4.6 [symbol] | 0.49 [symbol] | 4.51 [symbol] | 0.32 [symbol] | −0.25 [symbol] | 0.799 [symbol] |
| | RT [symbol] | 4.45 [symbol] | 0.5 [symbol] | 4.39 [symbol] | 0.49 [symbol] | −0.61 [symbol] | 0.542 [symbol] |
| | RP [symbol] | 4.55 [symbol] | 0.49 [symbol] | 4.42 [symbol] | 0.65 [symbol] | 0 [symbol] | 1 [symbol] |
| | RO [symbol] | 4.5 [symbol] | 0.67 [symbol] | 4.39 [symbol] | 1.07 [symbol] | −0.58 [symbol] | 0.559 [symbol] |

At this time, since the value of P (a meaningful probability value according to statistics) is reliable when being equal to or smaller than 0.05, it could be understood that the used frequency in the gamma band can be used for determining the degree of dementia.

In addition, the dimensional complexity of the brain wave signals measured in Equation 3 is compared with a threshold dimensional complexity defined as a minimal value of the dimensional complexity (S40), and then, the diagnosed person is determined as a normal person if the measured dimensional complexity is greater than the threshold dimensional complexity (S50).

In addition, if the measured dimensional complexity is smaller than the threshold dimensional complexity as a result of the comparison (S40), the diagnosed person is classified into a dementia-suspected group. Also, for the diagnosed person classified into the dementia-suspected group, re-determination is performed using at least one of DOI, MMSE, and CDR information, which determine the degree of dementia in the gamma band, which is stored in the memory unit 50 and calculated in an existing method (S60).

For reference, DOI, MMSE, or CDR information for determining the degree of dementia in the gamma band may be expressed as in the following Table 5. It is stored in the memory unit 50 in advance.

TABLE 5

| [symbol] | [symbol] | LF [symbol] | LT [symbol] | LP [symbol] | LO [symbol] | RF [symbol] | RT [symbol] | RP [symbol] | RO [symbol] |
|---|---|---|---|---|---|---|---|---|---|
| Gamma [symbol] | DOI [symbol] | −0.04 [symbol] | 0.22 [symbol] | 0.09 [symbol] | −0.21 [symbol] | −0.28 [symbol] | 0.13 [symbol] | −0.08 [symbol] | −0.05 [symbol] |
| band [symbol] | MMSE [symbol] | −0.01 [symbol] | 0.27 [symbol] | 0.17 [symbol] | −0.30 [symbol] | 0.04 [symbol] | 0.49* [symbol] | 0.27 [symbol] | 0.29 [symbol] |
| | CDR [symbol] | −0.05 [symbol] | −0.03 [symbol] | −0.31 [symbol] | 0.27 [symbol] | −0.01 [symbol] | −0.39 [symbol] | −0.43* [symbol] | −0.50 [symbol] |
| Whole [symbol] | DOI [symbol] | 0.22 [symbol] | 0.36 [symbol] | −0.05 [symbol] | −0.01 [symbol] | 0.09 [symbol] | −0.08 [symbol] | 0.10 [symbol] | −0.17 [symbol] |
| band [symbol] | MMSE [symbol] | 0.18 [symbol] | 0.22 [symbol] | 0.37 [symbol] | 0.25 [symbol] | 0.07 [symbol] | 0.27 [symbol] | 0.32 [symbol] | 0.32 [symbol] |
| | CDR [symbol] | −0.16 [symbol] | 0.01 [symbol] | −0.33 [symbol] | −0.46* [symbol] | 0.07 [symbol] | −0.02 [symbol] | −0.26 [symbol] | −0.26 [symbol] |

In addition, the dementia-suspected group is re-determined using DOI, MMSE, or CDR information (S60), and as a result, the diagnosed person is determined as a dementia-suspected patient if the diagnosed person has a value corresponding to a normal person (S70). Also, as a result of the re-determination (S60), the diagnosed person is finally determined as a dementia patient if the diagnosed person has a point corresponding to dementia (S80).

It is noted that although the technical spirit of the present invention described above is specifically described in the preferred embodiments, the aforementioned embodiments are for illustrative purposes and not to limit the present invention. In addition, it could be understood that those skilled in the art can make various modifications and changes thereto within the scope of the invention defined by the claims. Therefore, the true scope of the present invention should be defined by the technical spirit of the appended claims.

1. An apparatus for dementia diagnosis through brain wave analysis, comprising:
a measurement unit with electrodes having a plurality of channels for measuring brain wave signals;
an amplification unit for amplifying the brain wave signals measured by the measurement unit;
a dementia diagnosis unit for measuring dimensional complexity or the generation degree of synchronized brain wave signals on a specific frequency or in a specific frequency band based on the amplified brain wave signals; and
a dementia determination unit for diagnosing dementia based on the dimensional complexity or the generation degree of synchronization measured in the dementia diagnosis unit.

2. The apparatus as claimed in claim 1, further comprising a memory unit for providing specific frequency information or specific frequency band information for measuring the synchronization in the dementia diagnosis unit, and providing at least one of MMSE (Mini Mental Status Examination), GDS (Global Deterioration Scale), DOI (Duration of Illness), and CDR (Clinical Dementia Rating) information to the dementia determination unit.

3. The apparatus as claimed in claim 1, wherein the dementia diagnosis unit measures the synchronization of the brain wave signals using any one of GFS (Global Field Synchronization) and GSI (Global Synchronization Index).

4. The apparatus as claimed in claim 1, wherein the dementia diagnosis unit measures the dimensional complexity using brain wave signals in a gamma band among the brain wave signals measured by the measurement unit.

5. A method for dementia diagnosis through brain wave analysis, comprising the steps of:
measuring brain wave signals by performing potential measurement based on neuronal activation in the brain at fixed sampling intervals;
measuring the generation of synchronized brain wave signals on a specific frequency based on the measured brain wave signals; and
determining that a diagnosed person as a dementia patient if it is determined as a result of the measurement that no synchronized brain wave signal is generated.

6. The method as claimed in claim 5, wherein GFS and GSI are used for measuring the synchronization of the brain signals.

7. The method as claimed in claim 5, further comprising the steps of:
classifying the diagnosed person determined as a dementia patient into a dementia-suspected group;
re-determining brain wave signals of the classified dementia-suspected group by using any one of MMSE, GDS, and CDR information, which determine the degree of dementia on a specific frequency or in a specific frequency band according to an existing method; and
as a result of the re-determination, determining the diagnosed person as a dementia-suspected patient if it is determined that the diagnosed person shows a result corresponding to a normal person and finally determining the diagnosed person as a dementia patient if it is determined that the diagnosed person shows a result corresponding to a dementia patient.

8. The method as claimed in claim 7, wherein the specific frequency is at least one frequency of frequency components in alpha, beta1, beta2, beta3, and full bands when the MMSE information is used, and the specific frequency is at least one frequency of frequency components in alpha, beta1, beta2, beta3, and full bands when the CDR information is used.

9. The method as claimed in claim 5, wherein the method of measuring the synchronization of the brain signals comprises the steps of:
performing Fourier transform to the brain wave signals measured at an electrode of each channel to calculate a complex number value at a specific frequency (f);
making the calculated complex number value correspond to a complex plane to perform PCA (Principal Component Analysis) for distribution of points on the complex plane so that two eigenvalues (E₁ and E₂) are extracted;
applying the calculated eigenvalues (E₁ and E₂) to an equation
$$GFS ( f ) = \frac{\vert {E ( f )}_{1} - {E ( f )}_{2} \vert}{{E ( f )}_{1} + {E ( f )}_{2}}$$
to calculate a GFS value; and
determining that the measured brain wave signals are synchronized if the calculated GFS value is closer to 1, rather than to 0, and determining that the measured brain wave signals are not synchronized if the calculated GFS value is closer to 0, rather than to 1.

10. The method as claimed in claim 9, wherein the specific frequency is at least one of frequency components in theta, beta1, beta2, beta3, and full bands.

11. The method as claimed in claim 5, wherein the method for synchronization of the brain wave signals comprises the steps of:
calculating synchronization of electrode pairs corresponding to all brain wave signals measured on a specific frequency and combining the synchronization into a matrix format;
analyzing a pattern of the synchronization matrix through eigenvalue decomposition of the combined matrix to extract eigenvalues;
applying a greatest value (λ) of the extracted eigenvalues to an equation |GSI=(λ−ν)/(M−ν)| to calculate a GSI value; and determining that the measured brain wave signals are synchronized if the calculated GFI value is closer to 1, rather than to 0, and determining that the measured brain wave signals are not synchronized if the calculated GFI value is closer to 0, rather than to 1,
wherein M of the equation represents the number of the measured brain wave signals (which is equal to the number of electrodes), and ν is an average eigenvalue according to eigenvalue calculation using surrogate data.

12. The method as claimed in claim 11, wherein the specific frequency is at least one of frequency components in theta, beta1, beta2, beta3, and gamma bands.

13. The method as claimed in claim 11, wherein the method for calculating the synchronization for the electrode pairs uses at least one of coherence, phase coherence, and equal-time correlation.

14. A method for dementia diagnosis through brain wave analysis, comprising the steps of:
measuring brain wave signals by performing potential measurement based on neuronal activation in the brain at fixed sampling intervals;
measuring dimensional complexity in the gamma band based on the measured brain wave signals; and
as a result of the re-determination, determining a diagnosed person as a dementia patient if the measured dimensional complexity is greater than a threshold dimensional complexity at which dimensional complexity is defined to a minimal value.

15. The method as claimed in claim 14, further comprising the steps of:
classifying the diagnosed person determined as a dementia patient into a dementia-suspected group;
re-determining brain wave signals of the classified dementia-suspected group by using any one of MMSE, GDS, and CDR information, which determine the degree of dementia in a gamma band according to an existing method; and
as a result of the re-determination, determining the diagnosed person as a dementia-suspected patient if it is determined that the diagnosed person shows a result corresponding to a normal person and finally determining the diagnosed person as a dementia patient if it is determined that the diagnosed person shows a result corresponding to a dementia patient.

16. The method as claimed in claim 14, wherein the dimensional complexity (D2) is measured using an equation
$$D 2 = {lim}_{r -> 0} {lim}_{N -> ∞} \frac{log C ( r , N )}{log r} ,$$
where C(r,N) is defined using an equation
$$C ( r , N ) = \frac{2}{( N - W ) ( N - 1 - W )} {\sum }_{i = 1}^{N} {\sum }_{j = i + 1 + W}^{N} θ ( r - \vert {{x}^{->}}_{i} - {{x}^{->}}_{j} \vert ) ,$$
xᵢ and xⱼ are points of a path in a phase space, N is a data pointer number in the phase space, the distance r is a range surrounding each reference point xᵢ, and θ is a Heaviside function defined as 0 if x<0 and 1 if x≧0.
